(19) United States
(12) Patent Application Publication
(10) Pub. No.: US 2016/0206369 A1
(43) Pub. Date: Jul. 21, 2016

(54) LAPAROSCOPIC SUCTION DEVICE AND METHOD

(71) Applicants: Conor J Walsh, Cambridge, MA (US); BETH ISRAEL DEACONESS MEDICAL CENTER, Boston, MA (US); President and Fellows of Harvard College, Cambridge, MA (US)

(72) Inventors: Abraham Frech, Aliso Viejo, CA (US); Conor J. Walsh, Cambridge, MA (US)

(21) Appl. No.: 14/783,645

(22) PCT Filed: Apr. 10, 2014
(86) PCT No.: PCT/US2014/033668

§ 371 Date: Oct. 9, 2015

Related U.S. Application Data
(60) Provisional application No. 61/810,518, filed on Apr. 10, 2013.

(51) Int. Cl. A61B 18/14

(52) U.S. Cl. CPC A61B 18/1492; A61B 2218/002; A61B 2218/007

(57) ABSTRACT

The present invention relates to a surgical suction device that provides a controlled negative pressure to a surgical site. The device includes a tubular body with a distal absorbent material and a handle manually grasped by the user to control levels of negative pressure at the surgical site to adjust the flow rate of fluid during removal. Preferred embodiments allow switching between irrigation and suction and can provide for other treatment tools, including electrocautery.

### CROSS REFERENCE TO RELATED APPLICATION

This application claims priority under 35 U.S.C. §119 to U.S. Provisional Application No. 61/810,518, which was filed on Apr. 10, 2013, and is incorporated by reference in its entirety.

### BACKGROUND

To safely and efficiently perform surgery, surgeons use suction and irrigation to maintain a clear field-of-view. To achieve this view, suction devices can use negative pressure (suction) or a vacuum to remove fluid from an operating field such as the abdominal cavity. As negative pressure is applied directly to fluid within the abdomen (“direct suction”), surrounding tissue may be drawn toward a suction-device lumen, which can occlude the device lumen, thereby preventing further fluid removal, resulting in inefficiency and potential damage to tissue. To avoid these problems, surgeons may use sponges independently to absorb fluid or in conjunction with suction to remove fluid (“indirect suction”). Sponge insertion, however, may be time consuming, and sponges may further obstruct the field-of-view or may be unintentionally left in the body.

Surgeons depend on suction devices and irrigation to maintain adequate visualization. Current suction devices, however, are inefficient in many respects. For example, one concern with suction devices is the force imparted to tissue caused by the force at the openings of the suction device, which can result in occlusion. An occluded opening subsequently causes an increased negative pressure at any remaining openings. This increased negative pressure further attracts tissue and may eventually completely occlude the device, rendering it temporarily useless. Besides rendering the device useless, the trapped tissue can be damaged by the applied force.

In addition, another problem with suction devices , in particular laparoscopic, is that they exert negative pressure on the pneumoperitum, i.e., air used to obtain the visual field in laparoscopy, which tends to obstruct visualization. Suction devices allow surgeons to remove unwanted fluid and may be combined with other features such as irrigation to prevent the inefficiency of having to remove the suction device through a port and insert a separate irrigation device. The problem of inefficiency may even lead to frustration as surgeons attempt to prevent occlusion or dislodge the occlusion by retracting tissue away from the area to be suctioned or rapidly moving the suction device. This rapid, halting movement results in loss of time and a less efficient suction process.

Surgeons also use sponges to remove liquid from inside the body. In laparoscopic surgery, sponges are inserted through a port and moved into position with graspers. At times, surgeons will suction through a sponge or gauze. The act of inserting sponges has disadvantages, however, including inefficiency, the need to keep track of the number of sponges used and to make sure that all the sponges, which are not biodegradable and are a hazard to the patient if left behind, are removed at the end of surgery.

### SUMMARY

The present invention is directed to devices and methods for surgical suction and includes a device that integrates absorbent materials into a suction device. A preferred embodiment uses both suction and sponge material to provide controlled negative force at different flow rates. The device can be made with an adjustable sponge attachment, which allows the surgeon to choose between direct suction, used for removing large amounts of liquid, and indirect suction through the sponge. This device enables manual and intuitive use by the surgeon with reduced risk to the patient. The device can be manufactured with engineered absorbent materials shaped and sized for insertion into body cavities during surgery. Alternatively, with certain embodiments, existing laparoscopic sizes, surgical sponges, and tubing can be used. The device permits both direct and indirect suction, as well as irrigation, thereby enabling multi-functional uses that are important in laparoscopic and other surgical procedures.

Laparoscopic or minimally invasive surgeries are increasing in prevalence due to the small incisions, which reduce adverse surgical complications and are generally preferred by patients. The relative youth of the field, however, and the limitations inherent to minimally invasive surgery mean that the field has a number of obstacles not present in the traditional open surgery. In laparoscopic surgery, ports are placed into the patient's abdomen via percutaneous insertion with one or more trocars. The ports generally range from 3 mm to 15 mm in size. These ports stabilize and allow instruments such as graspers, dissectors, cameras, energy devices, suction devices, electrocautery, staplers, clips needle drivers, and sutures to be inserted into a body cavity such as the abdominal or thoracic cavity.

The present invention includes a preferred embodiment that comprises a tubular suction device that combines sponge and suction techniques at first and second flow rates while optionally incorporating irrigation and/or monopolar energy. The first flow rate involves indirect suction in which flow is directed through an absorbent element that can safely contact tissue. A second, higher flow rate can be used when the risk of occlusion or damage to tissue is reduced. The device can be configured to have a plurality of higher flow rates, including two, three or more flow rates. The device is sized for use in various laparoscopic or minimally invasive surgeries. In addition, the devices of the present application may also be adapted for us in open surgeries (e.g. vascular, neurological, orthopedic and dental) or open surgeries. The devices can also be used with suction tubing (i.e. nasogastric tubes, chest tubes, and drains). A preferred embodiment of the invention includes a tubular body connected at a proximal end to a handle that is manually grasped by the user to operate the device.

A porous, liquid-transferring or absorbent material is able to move relative to a distal end of the tubular body. The liquid-transferring or absorbent material can be moved to extend distally from or retract along the tubular body and may contact tissue during suction, or may be placed in contact with fluid within the body. The material, such as a sponge, provides a first flow rate that is “indirect,” as the fluid must flow through the material to enter the cavity within the tubular body. The user can move the material into a second position such that the distal opening is coupled to one or more channels in the tubular body to provide a second, higher flow rate (i.e. “direct” suction) to remove larger amounts of fluid material, e.g., when it is not necessary to contact tissue that can obstruct the distal opening, when contacted tissue will not occlude the opening, and/or when more rapid removal of fluid (e.g., faster suction) is needed. The handle can include a plurality of actuators to control movement of the absorbent, fluid-transfer material, or a tube that can be moved distally through the materials, and operate the suction and/or irrigation features sequentially or in combination to provide improved visualization during surgical procedures. A preferred method of performing a surgical procedure in accordance with preferred embodiments of the invention comprises percutaneously inserting a port into a body cavity and then inserting a tubular portion of the suction device through the port to a surgical site. The user then actuates the device to remove a fluid at one or more flow rates using direct or indirect suction modes to clear the field of view observed through a scope or other visualization device. The absorbent material can be a molded plastic polymer material with substantially uniform porosity.

The present application further provides devices and methods that allow indirect suction in surgical procedures. The devices can include an absorbent material connected to a flexible suction source. The device can also be used as a negative pressure drain device after surgery. The devices can be used to provide indirect suction or allow sponge contact with tissue, e.g. through a laparoscopic port, while helping to ensure that surgical sponges are not accidentally left behind and/or provide even suction without clogging. The devices can be used through a variety of access ports, including single-lumen ports and/or multi-lumen ports. In addition the devices can be used for single-incision laparoscopic procedures surgery. In such procedures, an absorbent material can be positioned within the incision with an attached suction tube such that suction can be applied during most or all of the procedure.

This continuous suction can reduce the risk of seroma formation after closure of the wound. The use of continuous surgical suction can utilize a flexible tube with shape memory characteristics such that the distal end of the tube can be bend by the user into a nonlinear or curved position so as not to obstruct the procedure or the visualization thereof by the user. The tube having a shape memory material, or components having such characteristics can have a plurality of holes or apertures such that the suction force can be distributed over a larger area. The absorbent material can have a generally cylindrical shape that covers the holes and the distal opening of the tube. The absorbent material can be permanently affixed to the tube with an adhesive, or can be detachable, where the user can mechanically attach and detach a sponge with a mechanical connector.

### BRIEF DESCRIPTION OF THE DRAWINGS

FIG. 1 is a perspective view of the device in accordance with certain embodiments of the invention;

FIGS. 2A-2D illustrate distal end components for fabrication of a tubular body in accordance with the invention;

FIG. 3 depicts a handle trigger in accordance with the invention;

FIGS. 4A-4E illustrate assembly of a valve system within the handle;

FIG. 5 illustrate handle assembly components in accordance with a preferred embodiment of the invention;

FIG. 6 is a graphical illustration of flow rate using direct and indirect suction in accordance with the invention;

FIGS. 7A-7B are illustrate suction port clogging and the prevention thereof in accordance with the invention;

FIGS. 8A-8E illustrate a further embodiment of an adjustable suction endoscopic device;

FIGS. 9A-9E illustrate further embodiments of an adjustable suction devices;

FIGS. 10A-10C illustrate a further preferred embodiment of the invention of an adjustable suction device.

FIG. 11A is a perspective view of a surgical device including an electrosurgical instrument, according to certain embodiments.

FIG. 11B is a perspective view of a surgical device including an electrosurgical instrument and having an outer absorbent material removed, according to certain embodiments.

FIG. 11C is a cut-away perspective view of a surgical device including an electrosurgical instrument, according to certain embodiments.

FIG. 11D is an open perspective view of a handle region of the device of FIG. 11C.

FIG. 11E is a perspective view of a surgical device including an electrosurgical instrument and having an absorbent material element retracted, according to certain embodiments.

FIG. 11F is a perspective view of a surgical device having an absorbent material element retracted and an electrosurgical instrument exposed, according to certain embodiments.

FIG. 12 illustrates a surgical device for providing indirect suction during laparoscopic procedures.

FIG. 13 illustrates use of the device of FIG. 12 along with a laparoscopic access port and one or more additional laparoscopic instruments.

FIG. 14 illustrates use of the device of FIG. 12 along with another type of laparoscopic access port and one or more additional laparoscopic instruments.

FIG. 15 illustrates use of the device of FIG. 12 along with a single-lumen laparoscopic access port and one or more additional laparoscopic instruments.

### DETAILED DESCRIPTION OF EXEMPLARY EMBODIMENTS

According to various embodiments, surgical devices are provided. As discussed below, the devices can include an elongated body having a proximal end and a distal end, the elongated body being configured to pass through a laparoscopic or minimally invasive surgical access port to assist in minimally invasive surgeries (e.g., laparoscopic surgery, thorascopic surgery, or any other similar minimally invasive surgery) or open surgery.

The devices can include a handle attached to the proximal end of the elongated body, the handle having a port for coupling to a negative pressure source or other source (e.g., irrigation source). In use, a surgeon or other operator (e.g., robotic surgical system controlled by a surgeon) will grasp and manipulate the handle and, as discussed in more detail below, may operate various components and/or functions of the device through controls located on or passing through the handle. As an example, the handle may have actuators for controlling the negative pressure source, supplying irrigation fluids, or controlling therapeutic instruments such as an electrosurgical instrument, which may be incorporated into the device.

Furthermore, as explained in greater detail below, the devices of the present application can comprise an absorbent material mounted on a portion of the elongated body and a centrally located channel passing through at least a portion of the elongated body. The absorbent material can be positioned over a distal opening in the channel and extend proximally along the tubular body. The absorbent material and the channel can be positionable in a first configuration to provide a first fluid flow rate through the absorbent material and the channel, and may also be positionable in a second position to provide a second fluid flow rate primarily through the channel As such, in the first configuration, the devices of the present application can be used to provide indirect suction through the absorbent material, and in the second configuration, can provide direct suction at a higher rate than the first rate. Of note, either configuration may provide fluid flow through both the absorbent material and the channel.

In certain embodiments, surgical devices comprising a tubular body having a proximal end, a distal end, and a centrally located fluid passage extending from an opening at the distal end and at least partially through the tubular body towards the proximal end are provided. The devices can again include a handle attached to the proximal end of the tubular body, and the handle can have an actuator and a port for coupling to a negative pressure source. An absorbent material can be positioned over a distal opening in the channel and extend proximally along the tubular body. In addition, the absorbent material can be movable between a first position and a second position, wherein in the first position the absorbent material is positioned along a fluid path through the opening at the distal end of the tubular body to provide a first fluid flow rate through the absorbent material and fluid passage, and a second position to provide a second fluid flow rate through the fluid passage of the tubular body without passage of fluid through the absorbent material.

In addition, methods of treatment are provided. The methods can permit suctioning of fluid from a surgical site. The methods can include positioning a suction device within a surgical site, the device including a tubular body with a fluid channel and a movable absorbent material. The absorbent material can be positioned over a distal opening in the fluid channel and can extend proximally along the tubular body. The methods can further comprise applying negative pressure to the absorbent material to remove fluid from the surgical site through the absorbent material and the tubular body fluid channel at a first flow rate, and adjusting a configuration of the absorbent material and the tubular body to provide fluid communication between a distal opening in the tubular body and the fluid channel of the tubular body to remove fluid from the surgical site at a second flow rate through the fluid channel that is higher than the first flow rate.

As used herein, “absorbent material” will be understood to refer to any material that absorbs fluids when contacted therewith. Absorbent materials can include porous materials such as clothes, sponges, or fabrics, and will generally include fluid paths therethrough to permit suction by applying negative pressure to the material.

Additional, details of various embodiments of the devices and methods disclosed herein are provided below.

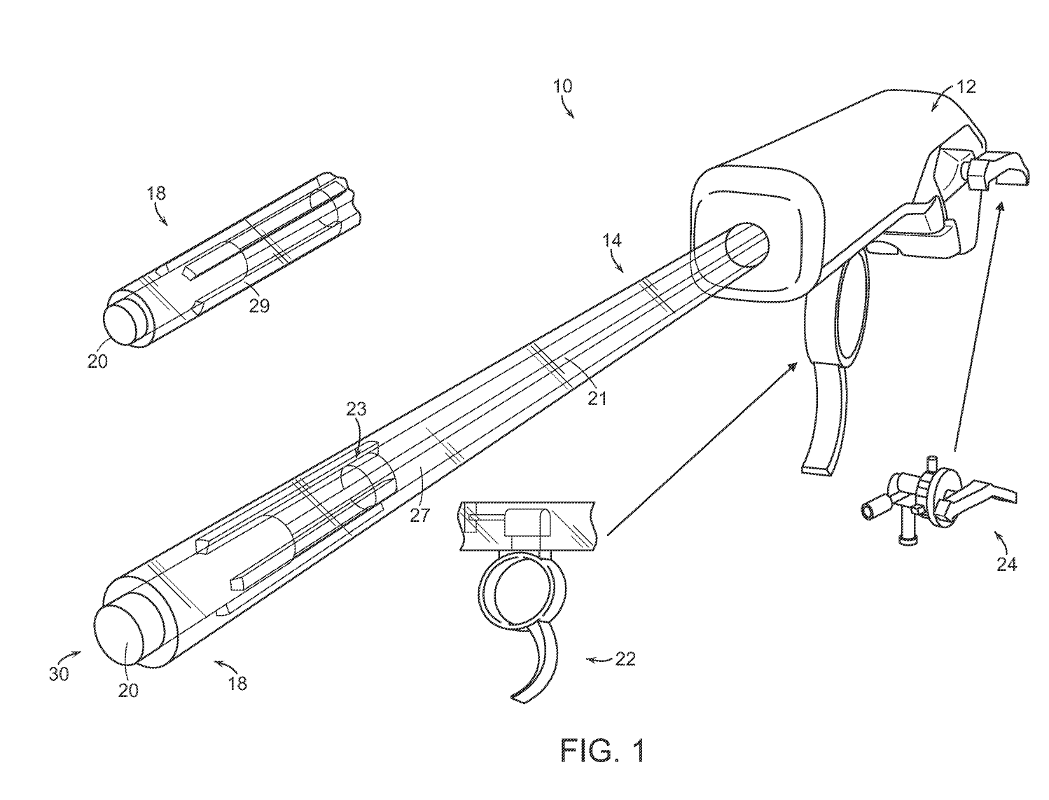

FIG. 1 illustrates a device 10, according to certain embodiments. In one embodiment, direct suction can be provided using channels 29 that provide a fluid pathway that bypasses a porous fluid transfer material 20. The bypass can comprise a number of veins or channels etched into the side of the tube. The material 20 can be actuated by a user with a handle to select either direct or indirect suction by moving the material 20 relative to the distal end 18 of the tube. The device 10 is shown in FIG. 1 where a handle 12 is attached to a tubular body 14. The distal end 18 has enclosed channels 29 and a fluid transfer material such as a sponge 20. The handle 12 can have a trigger 22 and a valve 24 to control operation of the suction and irrigation procedures. A sponge 20 can be mounted on a sliding element 21, and the device 10 can include a stopper 23 that forms a fluid tight seal with the inner wall surrounding the central channel 27 of the tubular body 14. The stopper 23 can thus operate to occlude the flow of fluid through the tubular body 14.

The sponge 20 can be mounted to the sliding element 21 (e.g., a tube or rod), which moves longitudinally within the tubular body 14 when actuated by the user with the handle 12 as described hereinafter. The sliding element 21 undergoes movement within the central channel 27 to provide relative movement between the sliding element and the tubular body 14 that can reposition the sponge 20 to a position that is proximal or to a distal opening between the annular channels 29 and the central channel 27. As such, when distal to the openings of the annular channels 29, as suction is applied to the channels 29, fluid being drawn into the device must pass through the sponge 20, thereby providing indirect suction. And, when the sponge 20 is proximal to the opening of the annular channels 29, fluid may be suctioned into the device through a distal opening 30 and into the channels 29 without passing through the sponge 20, thereby permitting direct suction that does not pass through the sponge 20.

For a given pressure difference between two sides of a porous material, the fluid goes through that material from a point of high pressure to a point of low pressure. Darcy's (discharge) velocity is defined as the amount of the fluid transferred per unit area of the cross section per unit time. According to Darcy's law, discharge velocity, q, is proportional to the pressure difference, namely

$$q = \frac{k}{μ} \frac{{p}_{2} - {p}_{1}}{L}$$

where k is permeability, and μ the viscosity, and L the distance between two points that pressures are applied. It is worth noting that q is not the velocity of fluid particles. To find the velocity of fluid particles, q should be multiplied by the porosity of the absorbent material. The objective is to increase q as much as possible. Because p₂-p₁ and viscosity of the fluid (here blood) are fixed, to increase q, a material with high permeability can be used and the distance of the bypass from the tip of the suction can be reduced. If this distance is very small, however, it will ineffectively prevent direct suction and may still become clogged when inserted into tissue.

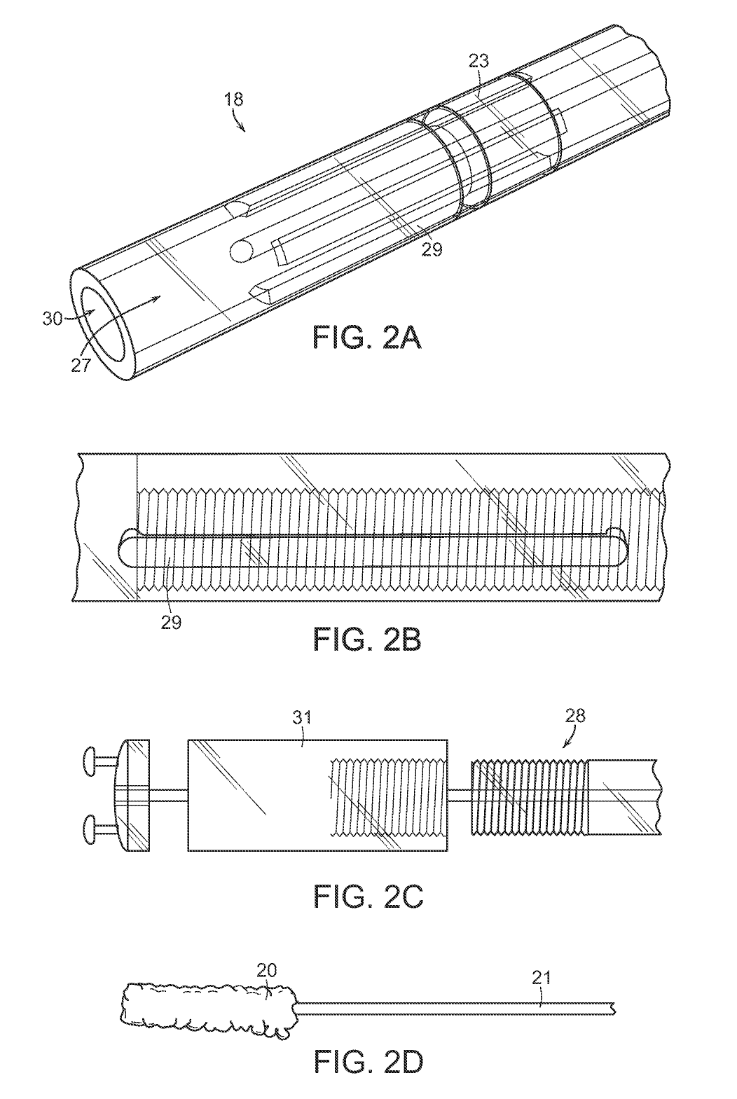

Using a device 10, as illustrated in FIG. 1 and FIGS. 2A-2D, suction was applied to one end of the material (sponge 20) while the other side was touching the surface of the fluid, and the amount of fluid sucked in versus time was measured to determine the change of volume of the fluid against time. Diaper material and sponges are very good absorbent materials, however, these materials typically cannot transfer fluid efficiently from one point to the other. Less densely packed gauzes can transfer the fluid more quickly. A single surgical sponge was found to be sufficiently permeable and has been used in conjunction with a preferred embodiment of the invention.

By optimizing the length and type of medical-grade gauze to be used, for example, the tube includes openings such as slits along the tube serving as the bypass system, as opposed to using tubes that project out from the side of the device. A tube and/or the absorbent material can be 3D printed or molded and has the bypass system such as veins or channels along the edge of the surface. Heat shrink tubing, or other outer tube can be positioned around the surface of the tube to enable flow.

In one embodiment, both suction and irrigation functions can be integrated with a handle. There were tubes in the back of the device with appropriate barbs to connect to normally sized suction and irrigation tubing found in hospitals. Adjustable valves can be included in the ports to both sources to enable manual adjustment of negative pressure for suction and positive fluid pressure for irrigation. Separate tubes can be used within the device to simultaneously provide irrigation fluid delivery and removal. These source connector tubes are connected to a valve inside the device that can switch between suction and irrigation. The output of this valve was connected using flexible tubing to the end of the main suction cylinder. This cylinder was made out of polycarbonate material, chosen for its strength and because it is a clear plastic. On the distal end of the cylinder, there was a “stopper” where the tube was no longer hollow, and the veins that ran down the edges of the tube to form the bypass. The veins were created by etching the side of the tube in four places and using a shrink-heat tube around the outside to create a seal. Holes were drilled on either end of the veins to form distal and proximal openings to fluidly connect the bypass above and below the stopper a hole extends through the stopper was a hole so that a rod can be inserted with the sponge on the distal end. This rod or tube 21 can be made of steel for strength and durability, and also using a rigid plastic material. The proximal end 28 of the cylinder (tubular body 14) was threaded, and a separate piece of polycarbonate handle 31 was screwed onto the proximal end 28. The handle 12 can have a hexagonal cross section to have an easy access point for the tube from the valve to connect suction into the cylinder. A hole was drilled into one of the hexagonal faces for that access. At the end of the cap, there was a small hole through with the sponge's rod or tube protrudes. This hole had a slightly larger concentric hole to receive an o-ring. This o-ring was kept in place by a second hexagonal piece of the same size also with a central hole for the sponge rod. This second cap was screwed into the first using tapped holes and screws, sized 2-56, for example.

The device enables surgeons to both adjust the position of the sponge to alter the level of negative pressure, and consequently the flow rate, and alternate between suction and irrigation. A slightly angled handle with a trigger and a switch to control the two modules can be included. The three modules were the veins and sponge system, the trigger, and the function switch near the back of the handle. The veins or channels can have different lengths, thus enabling the user to select the number of channels in use and thereby provide a plurality of different direct suction rates.

The channel system allows for liquid to be sucked away along four slits formed in the side of tube, which are covered with an outer tube. The slits cut in the outside of the tube, so that liquid being sucked away from the distal end travels upwards first, around the bypass where the sponge is located, and back inside the tube where the vein leads back to the center lumen of the tube. In this embodiment, the sponge feature position affects the vein bypass system as in can be moved between internal and external positions. When the sponge is deployed or placed “out”, so that it sticks approximately 1-3 mm outside of the tube, negative pressure is directed through the sponge and draws liquid away, although at a slightly lower rate. Suction can still applied as the main means of liquid removal and the sponge acts as a buffer, protecting vulnerable tissue from high negative pressures and can also absorb fluid. However, the sponge can be retracted back inside, behind the veins, for situations when the surgeon needs to use direct suction. FIGS. 2A-2D show different parts of this module.

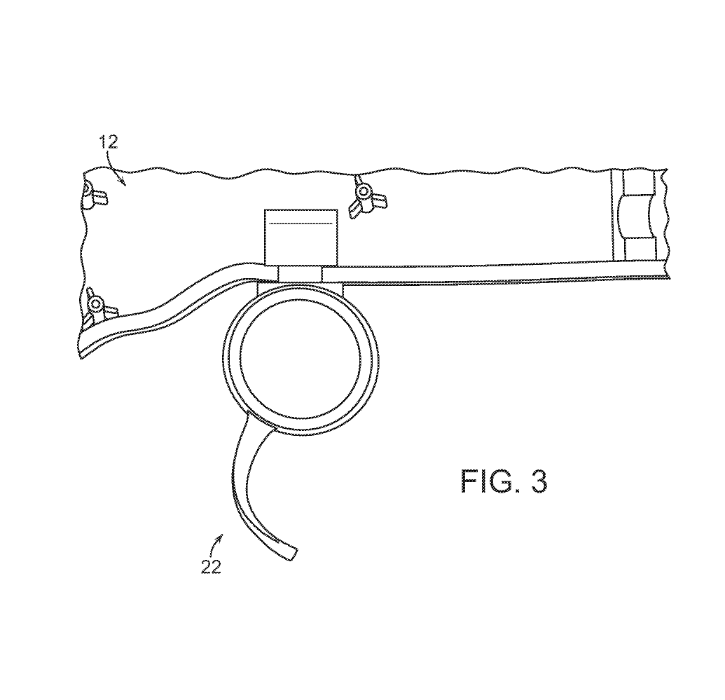

The trigger can be 3D printed or molded and connected directly to the end of the rod that the sponge was attached to. The inner diameter of the circle was 25.4 mm (1 inch) in order to enable users with large hands, even with surgical gloves, to adjust the sponge feature. This inner part of the circle was also configured with the computer and 3D printed to make it more comfortable for surgeons to use during long hours of surgery. The total length that this trigger could slide up and down and was also constrained to a displacement of 19.05 mm (0.75 in) in order to prevent the surgeon from accidentally pushing the sponge out too far and potentially injuring the patient. When slid completely back, the distal end of the sponge is proximal to one or more openings of the veins, so that the surgeon can use direct suction. FIG. 3 illustrates an actuator or trigger mounted to the handle.

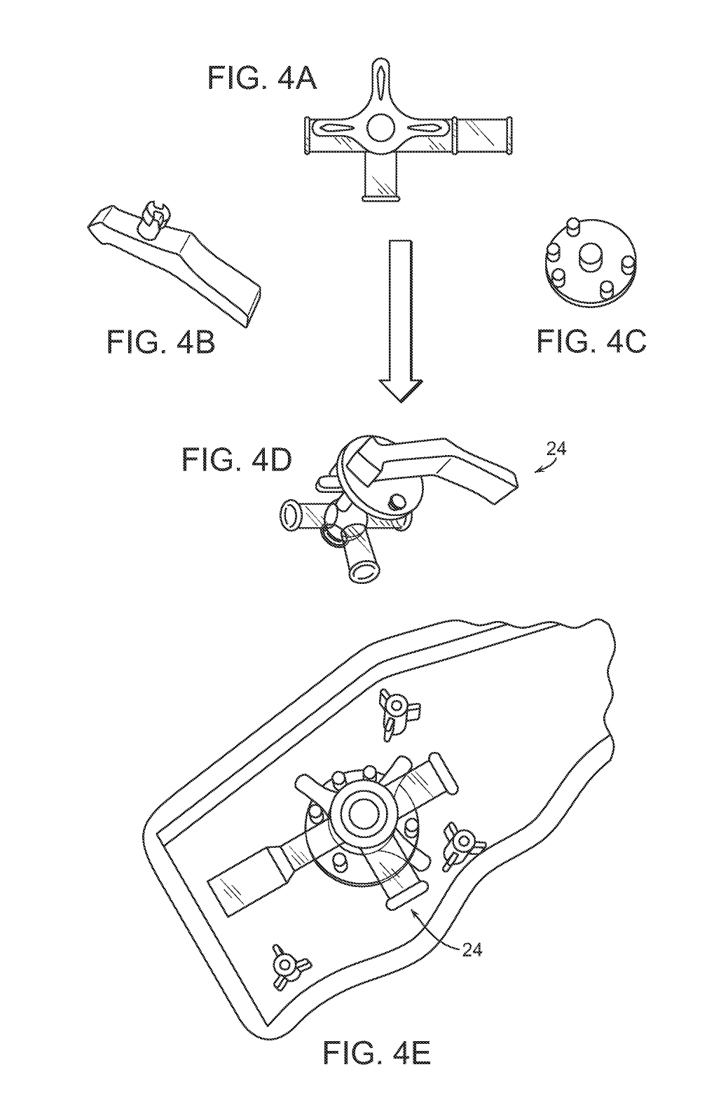

A second control module element comprises a switch to actuate the change from suction to irrigation. This was inserted through the side of the handle and had a number of features that pushed on the control valve to change from suction to irrigation. This switch element had an element on it that fit inside a feature on the housing that stopped the switch from turning more than 90 degrees so that the device can only switch from suction to irrigation and back. This also offers the benefit of being able to be turned 45 degrees, and thus be turned “off”—neither irrigation nor suction are being applied, and thus the surgeon can leave the device and focus his attention on other matters without fear that the surgical area will be filled up with water or that tissue will be damaged. The switch was made to contrast strongly with the handle color, so that the surgeon can easily see which feature is being used. FIGS. 4A-4E illustrates different parts of this module, including components of a valve 24 (FIGS. 4A-4D). In a further embodiment, both suction and irrigation can be deployed simultaneously using different delivery and removal channels.

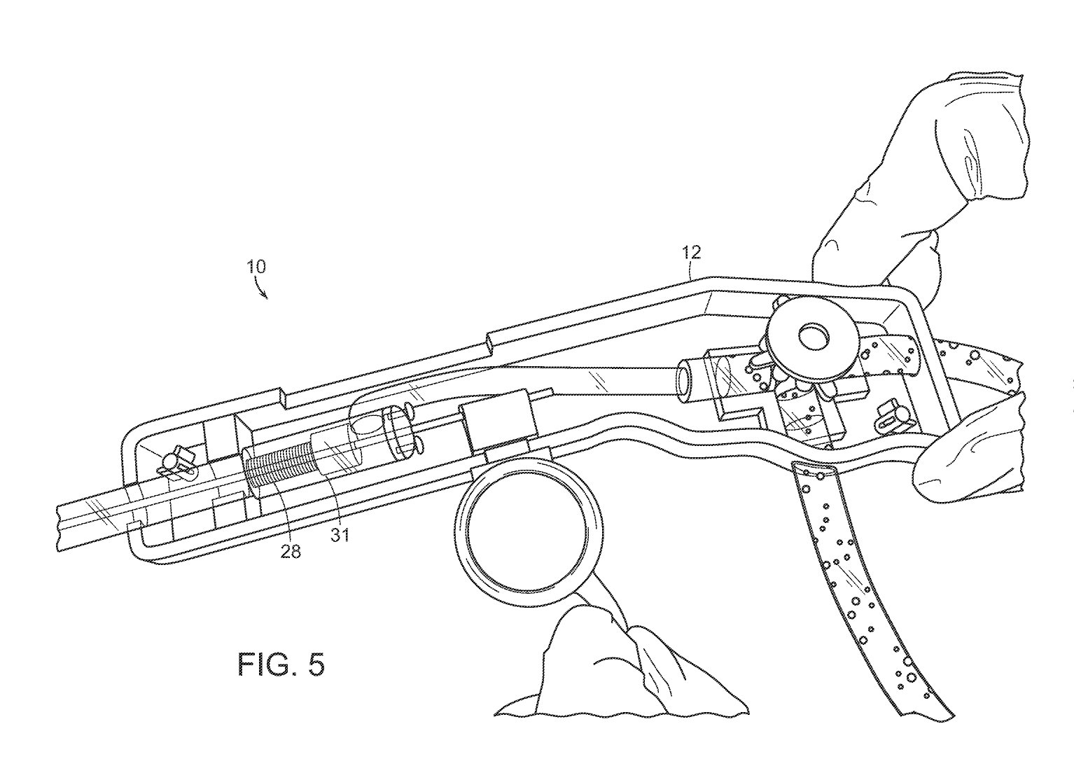

The embodiment of FIG. 5 successfully demonstrated suction effectively using a range of materials without undergoing excessive changes in flow rate from direct suction. The handle body is shown with one half removed to show the trigger coupled to a control rod entering the proximal end of the tube. A second actuator controls selection of suction or irrigation fluid being coupled to the internal cavity of the tube.

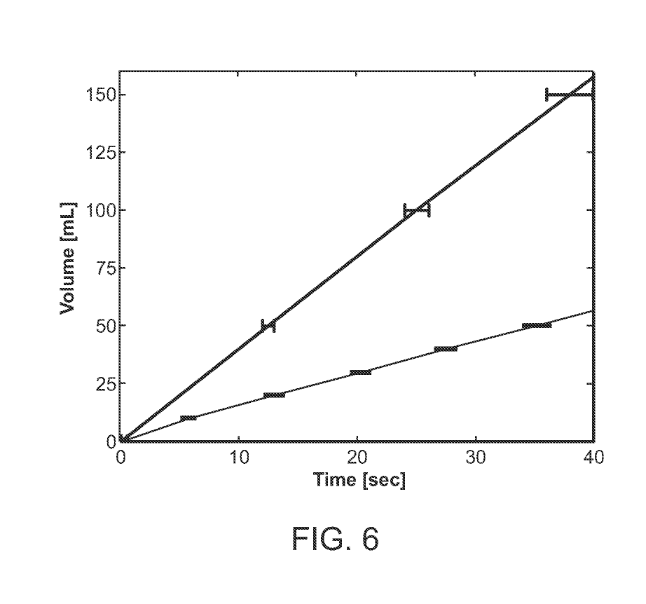

In this measurement, the volume of the fluid flow of this embodiment was measured over time for direct and indirect suction. FIG. 6 shows a linear relation for the flow rate. Although, in this embodiment, the surgeon has the option to switch between direct and indirect suction when it is safe, as it is shown by this method, the flow rate of the indirect case can be comparable to the flow rate of the direct suction. In FIG. 6, the error bars show the standard error in this embodiment. Generally flow rates below 2.5 ml/sec are provided by indirect suction and flow rates above this level are provided by direct suction. Different flow rate thresholds can be selected depending on the type of surgery, the flow characteristic of the fluid (blood, water, saline) and the type of tissue.

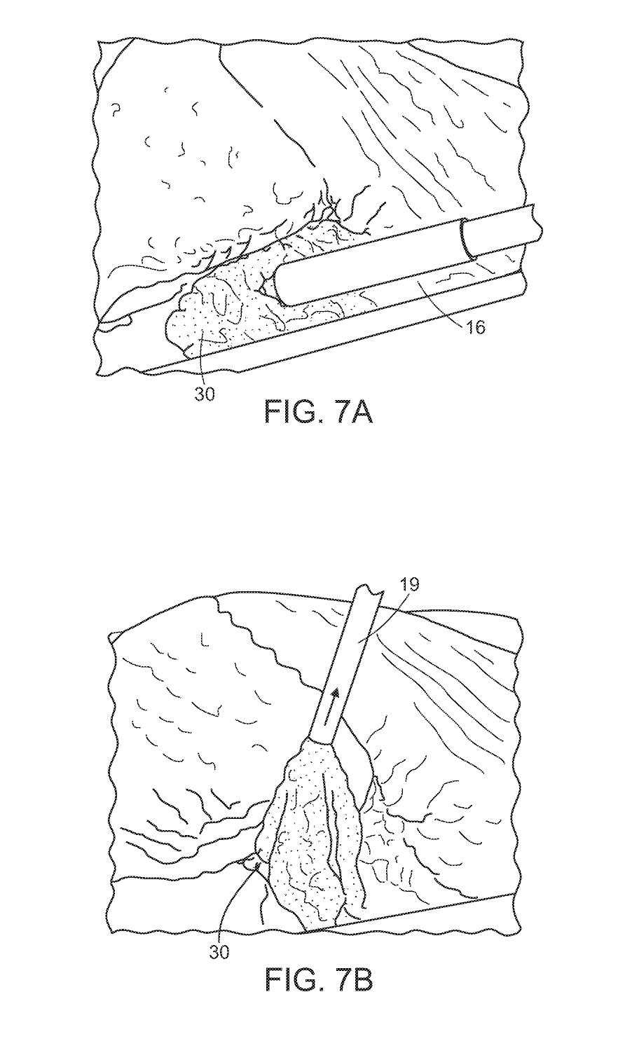

In order to show the advantage of the invention over the current surgical suction devices, a soft tissue sample was used and the device removed water with suction in contrast to a Frazier tip suction device. As shown in FIG. 7B, Frazier tip 19 suction got clogged by the fat 30, while the suction device of the present invention did not and was safe to the tissue, FIG. 7A.

Thus, the present invention combines direct suction or indirect suction at a plurality of selectable flow rates and irrigation. The surgeon has the option to push the sponge out of the tube to remove the blood on the soft tissues safely or pull back the sponge and switch between direct suction and irrigation at their convenience. Several measurements of flow rate show that this suction method does not get clogged, even with coagulated liquid, and can collect blood at a reasonable rate. Therefore, it is safe for soft tissues and saves time during surgery.

A preferred embodiment of the device can include a cautery tool added to the rod that contains the sponge. The cautery tool can be a monopolar or bipolar electrode that can be actuated by an actuator on the handle that is connected to an external power source. Along with this cautery function, a switch is included on the handle for controlling the electrode with a safety mechanism to ensure safety for the patient. The device can include a check mechanism, to ensure that irrigation cannot be used while the sponge is in the out position. This embodiment automatically pulls the sponge back (and out of the way) whenever the irrigation function is turned on. This ensures that the water sprayed by means of irrigation has enough force to wash away water from the field of operation, as opposed to being blocked by the sponge. The irrigation feature enables the water or other irrigation fluid to be delivered with sufficient force to enable the irrigation to effectively clean surgical areas. The tool is able to be comfortably used in one hand.

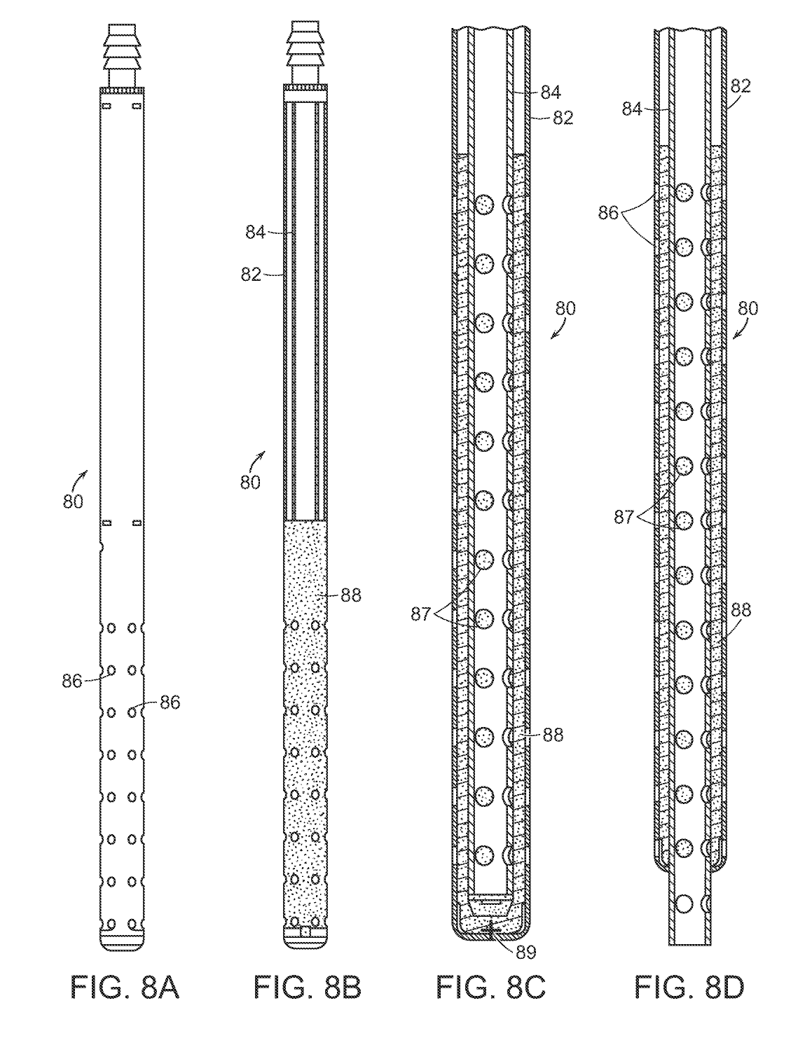

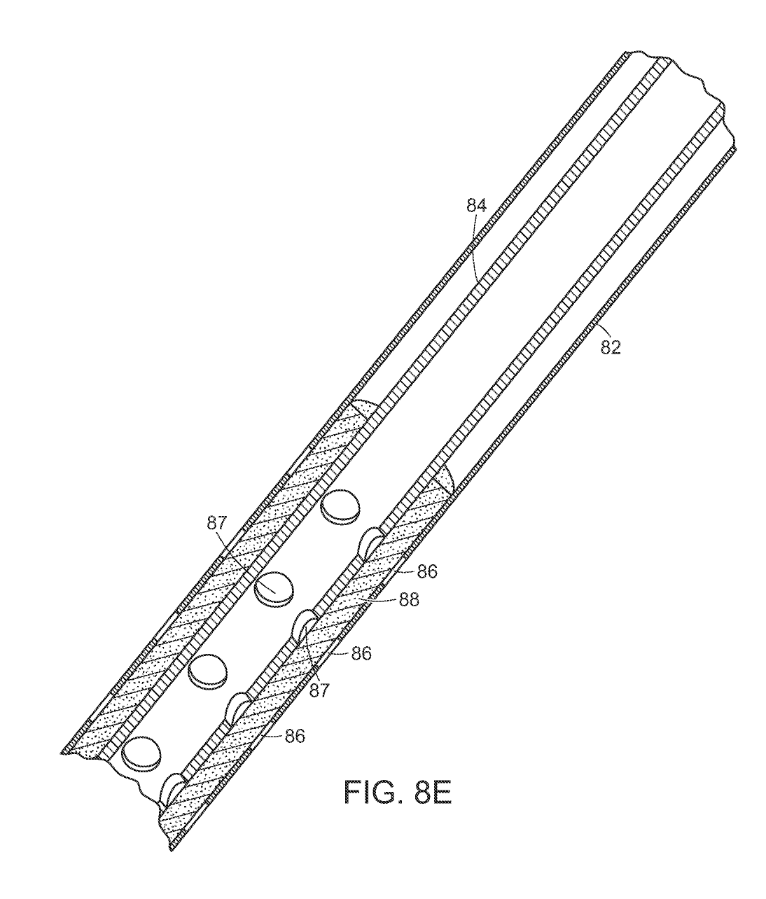

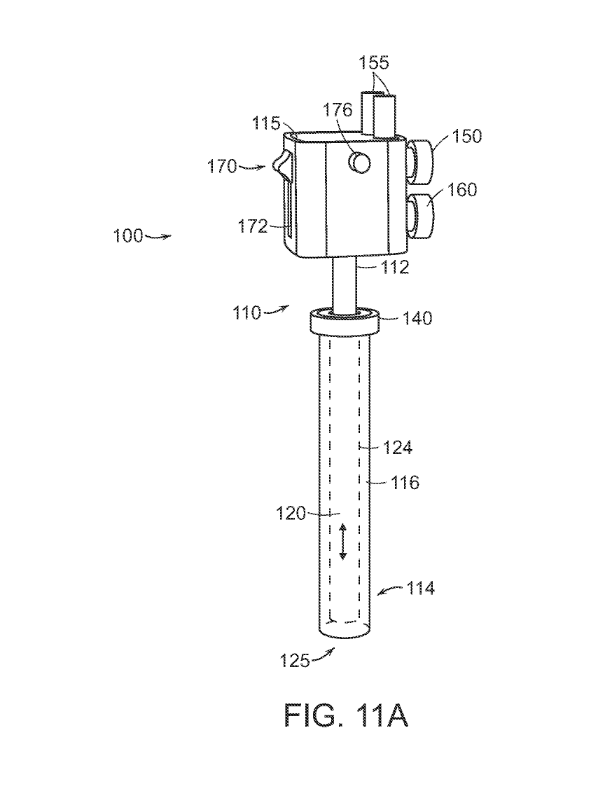

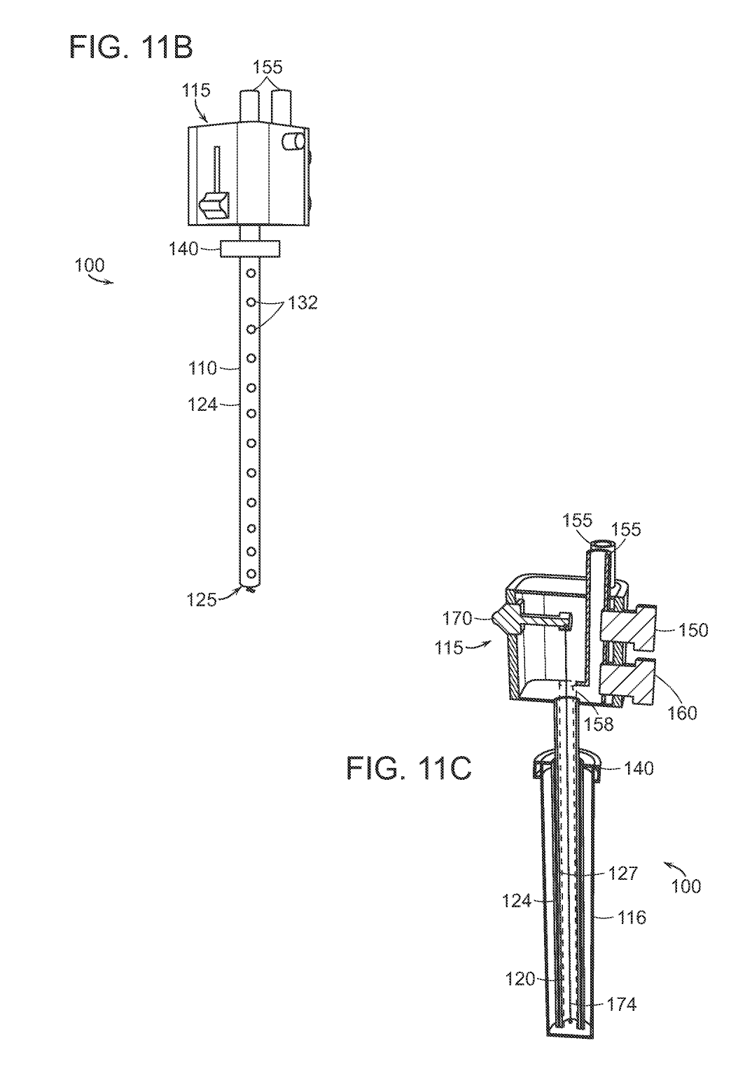

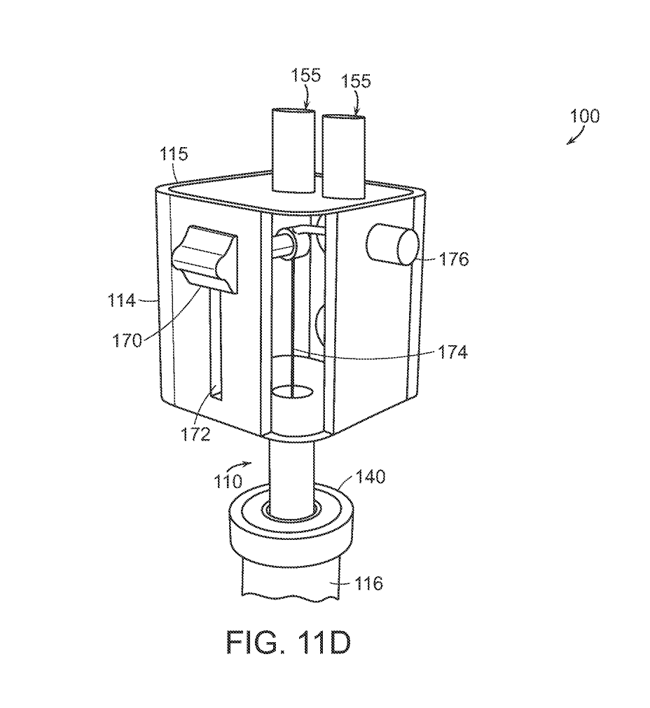

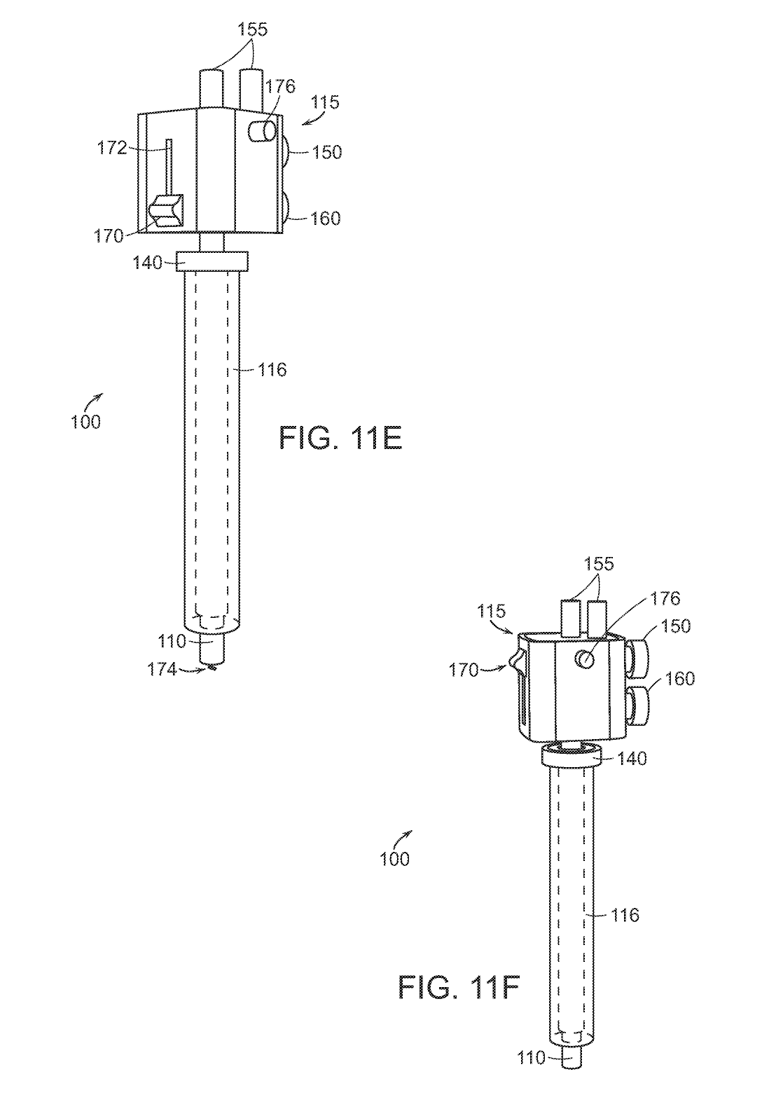

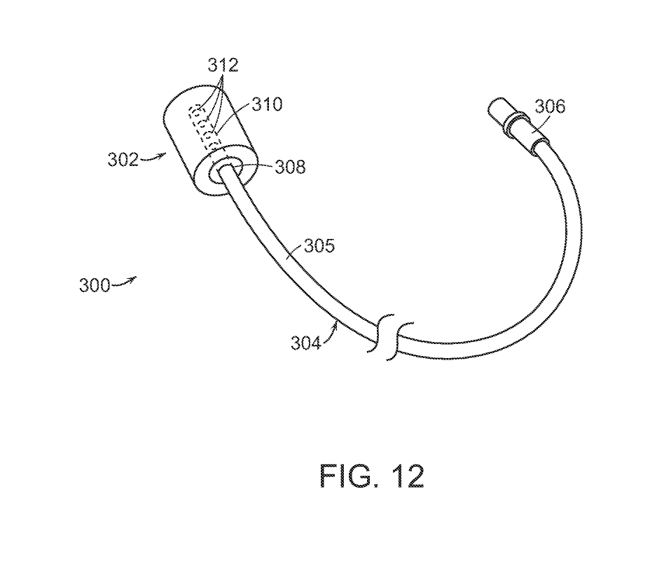

FIGS. 8A-11F further illustrate various embodiments of the devices and methods encompassed by the present disclosure. For example, FIG. 11A is a perspective view of a surgical device 100 including an electrosurgical instrument (described further below), according to certain embodiments. As shown, the device 100 can include an elongated body 110 (e.g., having a tubular shape) having a proximal end 112 and a distal end 114. As discussed previously the elongated body 110 (including components such as a absorbent material body 116 (discussed below)) can be configured to pass through a minimally invasive surgical access port to assist in minimally invasive surgeries (e.g., laparoscopic surgery, thorascopic surgery, or any other similar minimally invasive surgery), but can also be used in open or less minimally invasive procedures.

The device 100 can include a handle 114 attached to the proximal end 112 of the elongated body 112. In use, a surgeon or other operator (e.g., robotic surgical system controlled by a surgeon) will grasp an manipulate the handle and, as discussed in more detail below, may operate various components and/or functions of the device through controls located on or passing through the handle. As an example, the handle 115 may comprise actuators for controlling the negative pressure source, supplying irrigation fluids, or operative therapeutic instruments such as an electrosurgical instrument, which may be incorporated into the device.

Furthermore, as explained in greater detail below, the devices of the present application can comprise a absorbent material 116 mounted on a portion of the elongated body 100 and a fluid channel 120 passing through at least a portion of the elongated body 100. The absorbent material 116 and the fluid channel 120 can be positionable in a first configuration to provide a first fluid flow rate through the absorbent material 116 and the fluid channel 120, and may also be positionable in a second position to provide a second fluid flow rate through the fluid channel 120 without passage of fluid through the absorbent material 116. As such, in the first configuration, the devices of the present application can be used to provide indirect suction through the absorbent material, and in the second configuration, can provide direct suction at a higher rate than the first rate. In some cases, the absorbent material is over a distal opening 125 in the elongated body 110, and can extend proximally along the length of the body.

To provide indirect suction, the devices of the present disclosure can include an absorbent material 116 in a number of different locations or configurations with respect to the elongate body. For example, in various embodiments, the absorbent material is mounted at a distal end portion 114 of the elongated body 110, and also can extend along proximally along the elongated body 110. In various embodiments, the absorbent material 116 may extend proximally along the body 110 and completely surround the body 110 over a portion of the body length.

The absorbent material 116 can be formed from a variety of different materials. For example, suitable materials can be flexible and/or compressive to provide a relatively soft surface with which to contact tissue. In addition, suitable materials should have a sufficient permeability to allow fluid flow therethrough, and are absorbent to allow absorption of fluid without suction, if desired. In addition, the material should have sufficient density and resistance to fluid flow to allow a reduction in suction pressure across the material to produce a low to negligible level of negative pressure when suction is applied. Suitable materials may include, for example, a fabric, surgical gauze, sponges, and/or synthetic polymeric materials.

In various embodiments, the elongate body 110 can include an inner wall and an outer wall, as shown in the cross-sectional view of FIG. 11C. The inner wall 127 can comprise a first substantially rigid tubular member in which the channel 120 is formed, and the outer wall 124 can include a second substantially rigid material. The walls 124 can include one or openings so that suction applied to a channel 120 within the elongated body 110 is transmitted through the channel 120 and in the first configuration, through the absorbent material 116. In one embodiment, the elongated body 110 includes a distal opening 125, and the absorbent material 116 can be positioned in a fluid flow path through the distal opening 125.

In addition, the device 100 can include one or more other openings 132 extending through the walls 124, 127 of the elongated body 110 into the fluid channel 120 of the elongated body 110. For example, in some embodiments, as illustrated in FIG. 11B, the inner wall 127 can include one or more opening or holes 132 along its surface 124 and extending into the fluid channel 120. The openings or holes 132 can have a variety of sizes, shapes, or configurations, and can include elongated slits, circular openings, or openings of other shapes. In addition, the outer wall 124 can include openings along its surface (not shown) to allow fluid communication between the channel 120 and the outer region of walls 124, 127.

In order to switch the device (e.g., device 100) from an indirect suction mode having a first flow rate through the absorbent material 116 and fluid channel 120 and a direct suction mode through the fluid channel without passage through the absorbent material 116, the positions and/or configurations/spatial relationships between the channel 120, absorbent material 116, and opening(s) 125,132 may be altered. For example, in various embodiments, the absorbent material 116 is attached to outer wall 124. Further, the outer wall 124, and hence, absorbent material 116, may be moved from a distal position (FIG. 11A) to a retracted position (see FIG. 11F, which illustrates a perspective view of a surgical device including an electrosurgical instrument and having an outer absorbent material removed, according to certain embodiments) by sliding the outer wall 124 and absorbent material 116 proximally to expose a distal portion of the elongated body. The absorbent material 116 can have a region wherein the distal region comprises one or more flexible slits or flaps to allow passage of the tube 110 therethrough. In other embodiments, the tube 110 is configured to move, thereby having an annular wall that can extend through the absorbent material 116 to allow direction suction through the distal opening 125.

In some embodiments, the absorbent material 116 is not attached to the outer wall over openings 132 in the inner wall. That is to say, the absorbent material 116 may lie over a surface of the inner wall 124, and elongate body 110 may be formed with a single inner wall 127 and an absorbent material (no outer wall); or inner wall 124, outer wall 124 of short length at the proximal end of elongated body 110, and absorbent material 116 extending over the more distal region of the inner wall 127. In some embodiments, the absorbent material 116 and/or inner wall is attached to a proximal mobile member 140 that can be mechanically coupled to the handle 115 to allow movement of the absorbent material 116.

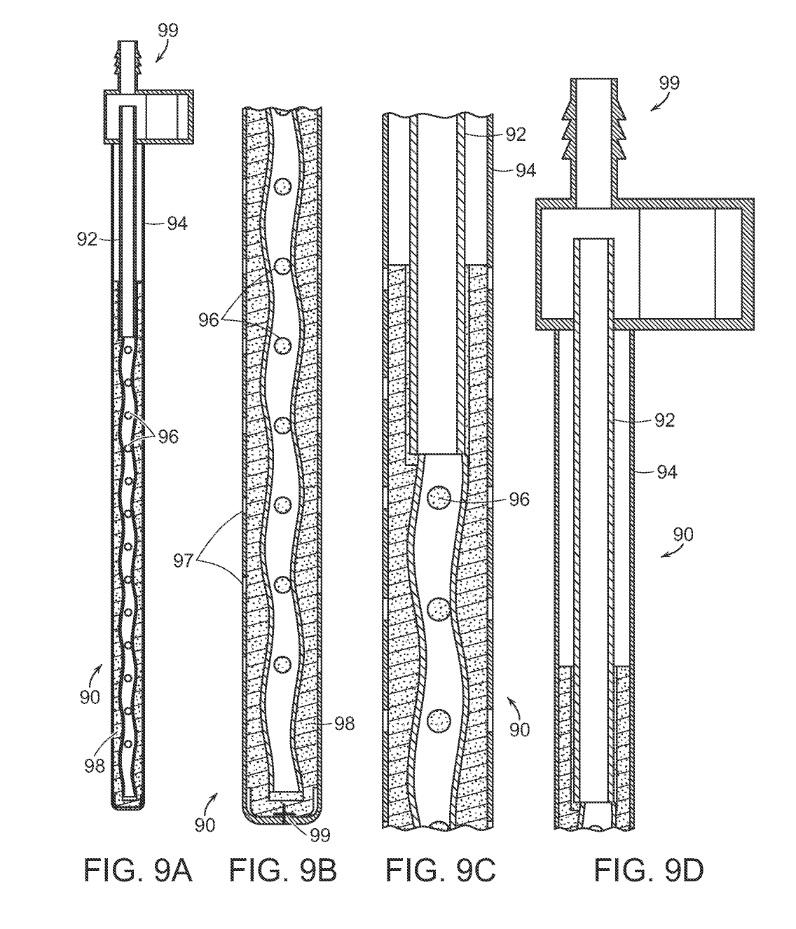

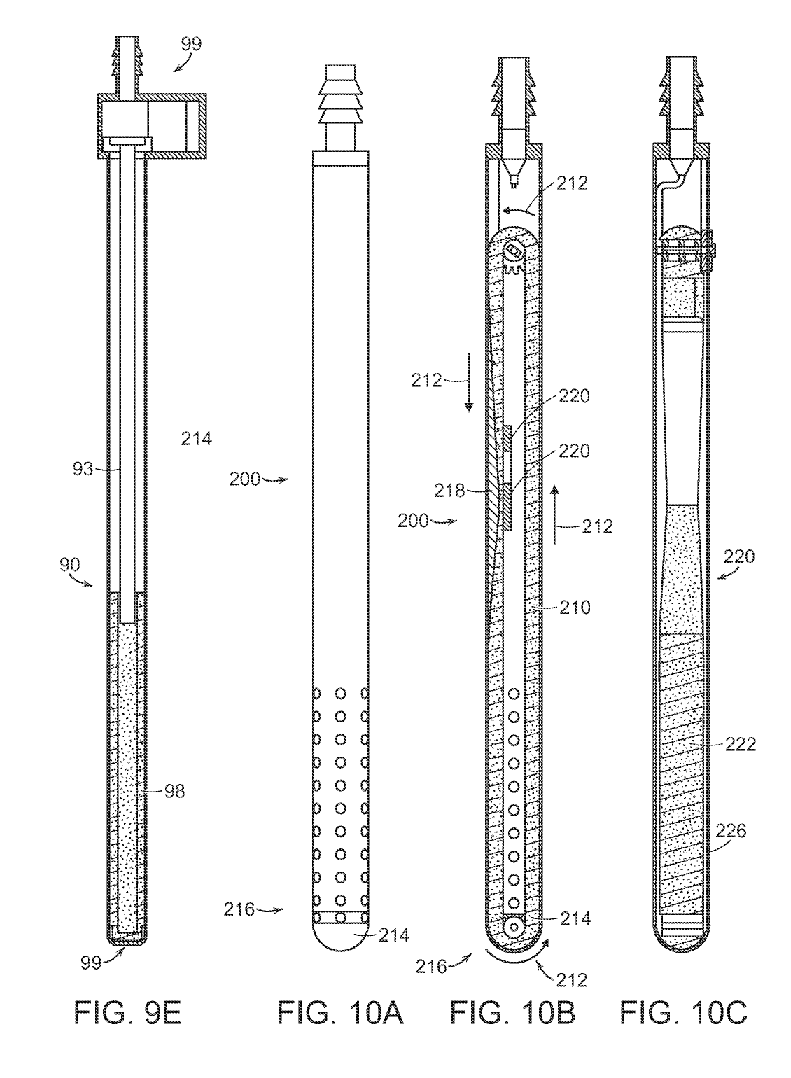

Further, in one embodiment, which is illustrated in FIGS. 8A-9E, the absorbent material 116 can be formed on an inner surface of the outer wall 124. As such, the absorbent material 116 will be positioned between the inner 127 and out walls 127, and both wall will include holes or openings to allow fluid flow. In such an embodiment, the absorbent material may be exposed at the distal end of the elongated body to allow an absorbent and/or more flexible portion that can safely contact delicate tissue.

As discussed above, the device 100 can include one or more treatment instruments that may be deployed through the distal opening 125. A variety of different instruments 174 can be selected, including for example an electrosurgical instrument, a cautery device, a grasping instrument, a cutting instrument, or fastening instruments (e.g., suturing, stapling, or clipping devices.

As an example, FIG. 11C is a cut-away perspective view of a surgical device 100 including an electrosurgical instrument 174, according to certain embodiments. FIG. 11D is an open perspective view of a handle region 115 of the device of FIG. 11C. As illustrated in FIG. 11D, the handle 115 can include a number of controls 150,160,170, for control of device operation. For example, the handle 115 can include an actuator 170 that can move along a path 172 for extending the electrosurgical instrument 174 from the opening 125, as illustrated in FIG. 11E.

The handle 114 can further include additional controls 150,160. For example, controls 150,160 can be configured to activate suction 150 or irrigation 160. Furthermore, the handle 115 can include connections 155,176. Suitable connections can include suction or irrigation connection 155 to deliver negative pressure and/or fluid, and electrical connections 176 for connection to instruments such as an electrosurgical instrument. In addition to controls 150,160 on the handle 114, the device 100 can include additional control elements. For example, the device may include an actuator on a foot pedal or similar device to allow control without use of hands. Furthermore, the controls 150,160 can be mechanically coupled to suction/irrigation tubing 156,157, which can be fluidly coupled with the channel 120. Further, the fluid connection can include a valve structure 158 to allow passage of instruments into the central lumen.

As illustrated in FIGS. 11A-11F, the device 100 include a centrally located lumen 120 for passage of fluids. In some embodiments, the device 100 can include additional lumens within the tubular body 110 for separate passage of material, including, for example, medications or irrigation independent of suction. The whole device can be disposable, or alternatively, the only certain components such as the sponge, tubing and valves may be disposable, leaving the handle free to be used in multiple procedures.

Another preferred embodiment is shown in connection with FIGS. 8A-8E in which two tubes, an outer first tube 82 and inner second tube 84 are used. Both tubes 82,84 have holes 86,87 to allow fluid movement. The outer tube 82 serves to maintain sponge location and the inner tube 84 provides the suction. The outer part of sponge 88 makes contact with outer tube 82 and absorbs fluid. The inner part of sponge 88 makes contact with inner tube 84 and suctions fluid. The inner tube 84 can move from inside the sponge 88 to outside the sponge 88. When the inner tube 84 is completely enclosed by sponge 88 it provides indirect suction. Alternatively, when the inner tube 84 is outside the sponge 88 it provides direct suction. The inner tube 84 can serve as a channel for irrigation or for placement of an electrocautery device.

FIGS. 9A-9E illustrate additional embodiments for the tubular elements. In these embodiments, (FIGS. 9A-9E) two tubes, outer first tube 94 and inner second tube 92. Both tubes 92,94 have holes 96,97 to allow fluid movement in which the outer tube 94 serves to maintain the location of the absorbent material or sponge 98. Inner tube shape is modified, ideally to a sine wave shape or other asymmetric shape along its longitudinal axis. The asymmetric shape can allow compression and decompression of the sponge 98 when the inner tube 92 is rotated. The outer part of sponge 98 makes contact with outer tube 94 and absorbs fluid and the inner part of the sponge 98 makes contact with inner tube 92. As the inner tube 92 rotates, the sponge 98 is compressed or decompressed and suctioned. The inner tube 94 can move from inside the sponge 98 to outside the sponge 98 similar to the embodiments described above. When the inner tube 92 is completely enclosed by the sponge 98, it provides indirect suction, and when the inner tube 92 is outside the sponge 98 it provides direct suction. As the sponge 98 extends the length of the tube 90, it can also be more fluid during removal. The inner tube 92 can serve as a channel for irrigation or for placement of an electrocautery device.

In addition, the top segment 99 of the device can include additional elements. For example, top segment 99 can be used for placement of a motor that actuates rotation of the inner tube 92. In some embodiments, the device 90 can include only a single tube 93. For example, FIG. 9E illustrates an embodiment without an inner tube.

In various embodiments, the sponge or absorbent material may cover a distal region 89,99 of the tube 80,90, and the inner tube may be extended through the distal region 89,99. Accordingly, the distal region 89,99 may include slits or openings, or may be otherwise configured to allow the inner tube to pass therethrough. It will be understood that each of the devices described with respect to FIGS. 11A-11F can be similar constructed.

FIGS. 10A-10C illustrate a preferred embodiment in which a sponge 210 is in the form of a shaped belt that rotates around a path 212 as shown. The bottom part of the sponge 214 at the distal end 216 absorbs fluid. As the sponge 210 travels along the path 212, the dry part of sponge 210 approaches the distal end 216. The wet part of the sponge 214 travels and gets compressed along the path at a compression region 218. Once compressed the fluid from the sponge 210 is transferred out and suctioned through openings 220 to a fluid channel. These embodiments enable selection of different flow rates that can be above or below one or more threshold flow rates that are selected by the user to more efficiently remove fluid and avoid device occlusion and injury to the patient.

Furthermore, FIG. 10C illustrates a similar concept, wherein a sponge 222 may be retracted or mobile within an outer tube 226. Accordingly, by moving the sponge 222, the sponge can be manipulated by movement over a shaped tube or compressing object to disrupt blockage should it occur or remove fluids.

The present application further provides additional devices and methods that allow indirect suction in laparoscopic or other minimally invasive procedures. The devices can include an absorbent material connected to a flexible suction source. The devices can be used to provide indirect suction or allow sponge contact with tissue, e.g. through a laparoscopic port, while helping to ensure that surgical sponges are not accidentally left behind and/or provide even suction without clogging. The devices can be used through a variety of access ports, including single-lumen ports and/or multi-lumen ports. In addition the devices can be used for single-incision laparoscopic procedures or other minimally invasive procedures.

FIG. 12 illustrates one surgical device 300 for providing indirect suction during laparoscopic procedures, according to various embodiments. As shown, the device 300 comprises an absorbent material 302, which can be attached to a flexible fluid conduit 304. Negative pressure and/or irrigation can be provided through the fluid conduit 304 using, for example, any standard connector 306 found at a proximal end of the fluid conduit 304.

As with absorbent material 116, described above, the absorbent material 302 can include a variety of different materials For example, suitable materials can be flexible and/or compressive to provide a relatively soft surface with which to contact tissue. In addition, suitable materials should have a sufficient permeability to allow fluid flow therethrough are absorbent to allow absorption of fluid without suction, if desired. In addition, the material should have sufficient density and resistance to fluid flow to allow a reduction in suction pressure across the material to produce a low to negligible level of negative pressure when suction is applied. Suitable materials may include, for example, a fabric, surgical gauze, sponges, and/or synthetic polymeric materials.

In addition, the absorbent material 302 can have a number of shapes. In one embodiment, the material 302 can have a cylindrical shape, which may assist in passing the material 302 through a surgical incision or access port, as described further below.

In order to provide efficient, even, and clog-free suction, the fluid conduit 304 and absorbent material 302 can be connected at a distal portion of the conduit 304. In particular, the distal portion 310 can pass part way into the absorbent material, and may optionally form a secure connection therewith. Further, the distal portion 310 can comprise group of openings 312 in fluid communication with the absorbent material and a central lumen 305 of the fluid conduit. The opening 312 can have a number, size, spacing, and shape selected to provide a desired level of uniformity of suction, while reducing a risk of clogging, as compared to use of a fluid conduit with a single opening at its distal tip.

In addition, in some embodiments, the absorbent material 302 and conduit 304 can be securely attached to one another at a connection region 308. The attachment can be formed at the time of manufacture and before use, at a surgical site before insertion of the material 302 into a surgical site, or after the material 302 is placed in a surgical site through an incision or access port. Further, various secure attachments can be used to ensure that the material 302 is not dislodged during use, and/or to allow removal of the material 302 and conduit 304 together through an incision or access port. Various connections can include threaded connections, a friction-fit connection, and/or an interlocking tab/slot system on respective parts of the material 302 and fluid conduit 304.

The devices disclosed herein can be used during laparoscopic and other minimally invasive procedures. Furthermore, the devices are suitable for use in single-incision or multi-incision laparoscopic procedures, and may be used with a variety of different laparoscopic access ports and/or in conjunction with other laparoscopic instruments. Furthermore, the device 300, described above, can provide a number of advantages in that it can be inserted through small incisions and/or through a laparoscopic port. In some procedures, the adsorbent material 302, while attached to the fluid conduit 304, can be inserted through a small incision or opening in an abdominal wall, and the conduit can be passed back through a port or allowed to remain in a position traversing the incision. As such, a relatively large sponge or adsorbent material can be used compared those usable in procedures requiring passage of the sponge or adsorbent material through a laparoscopic port into the abdominal cavity.

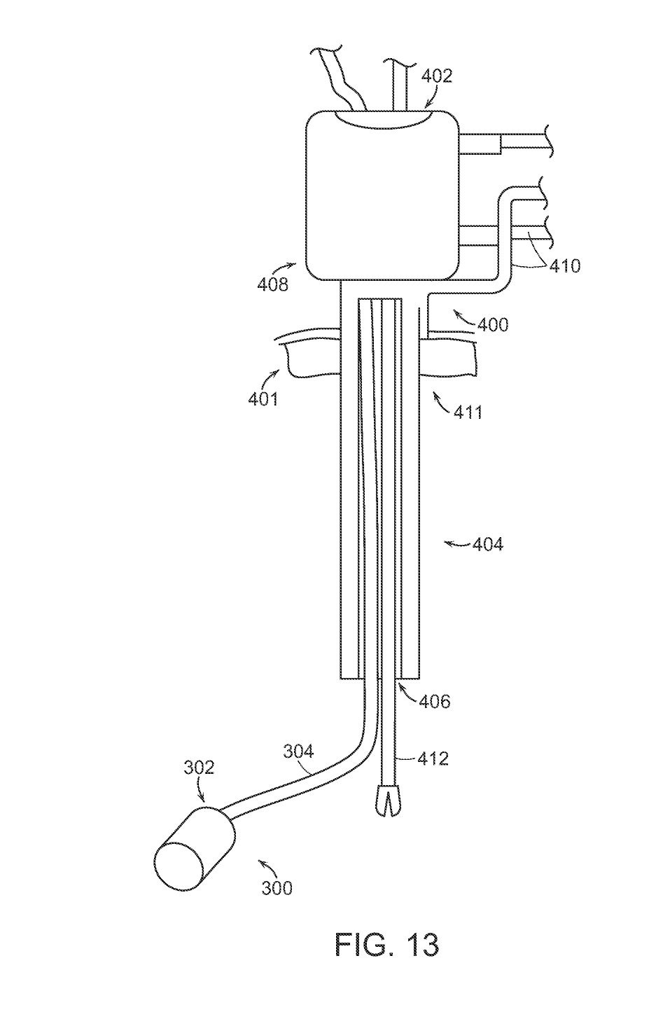

FIGS. 13-15 illustrate use of the devices 300 of the present application along with several types of access ports. FIG. 13 illustrates use of the device of FIG. 12 along with a laparoscopic access port 400 and one or more additional laparoscopic instruments 412. As shown, the port 400 includes a single pressurized chamber 408 configured to receive multiple instruments 300,412, which pass through a port lumen 411 and into an opening 406 that accesses the abdominal cavity. The port 400 will pass through an abdominal wall 401 through a single incision, thereby allowing use of multiple instruments through one port, and possibly providing the capability for single-incision laparoscopy.

In some methods, the device 300 provides suction and irrigation, as discussed above. Further, the device may be manipulated or placed into a desired suction/irrigation location using an instrument 412. The instrument 412, as well as additional instruments, can include a grasping instrument for manipulating the device, but can also include any other suitable laparoscopic tool.

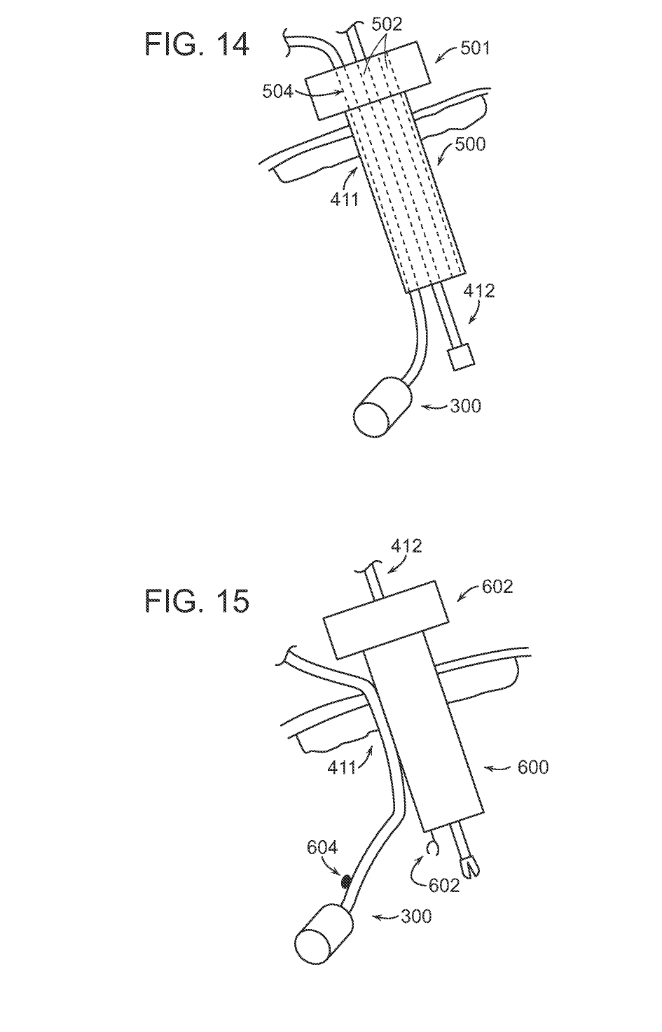

The device 300 can also be used along with a multi-lumen laparoscopic port 500, as shown in FIG. 14. Suitable multi-lumen ports can include a proximal housing 501 containing two or more lumens 504, 512, which receive instruments and allow passage through the port 500 into the abdomen. In some cases, the multi-lumen port 500 can include a small lumen 504 for receiving a relatively small fluid connection for the device 300, and one or more larger lumens 512 for receiving tools that have a larger area or require more space for manipulation. Use of a multi-linear port can enable continuous use of suction within the surgical field during the procedure.

Furthermore, in some methods, single-lumen ports 600, as shown in FIG. 15, may be used. When using a single-lumen port 600, the fluid conduit 304 of the device 300 may be passed through the incision 411 alongside the port, thereby allowing an instrument such as a grasping tool 412 to be passed through the port 600 at a housing 602. Alternatively, the adsorbent material 302 of the device 300 can be passed through the incision, and the fluid conduit 304 can be passed backwards through the lumen of the port 600, providing access to the device 300 through the single lumen port 600.

The systems may further include components for securing the device 300 in place when not in use, or alternatively, for continuous use during surgery, thereby preventing the device 300 or its associated tubing 304 from interfering with movement of other instruments or blocking the field-of-view. As examples, a port 600 (or 400, 500) can include a clamp, or other fixation component on its distal region or side wall. For example, a suitable fixation device may include a C-clamp 602. Alternatively, the adsorbent material 302 or fluid conduit 304 can include a clip or hook to allow the device to be secured to surrounding tissue. In addition, the port 600 can include additional clamps 602 or connecting elements, and the elements may be moveable by an operator. For example, the port can include a connecting element near its proximal end within the body cavity and/or one near its distal end. Further, the connecting elements may be configured to be extended, retracted, and/or rotated.

While the present invention has been described here in conjunction with certain preferred embodiments, a person with ordinary skill in the art, after reading the foregoing specification, can effect changes, substitutions of equivalents and other alterations to the systems and methods described herein. Each embodiment described above can also have included or incorporated therewith such variation as disclosed in regard to any and all of the other embodiments. Thus, it is intended that the scope of the claims granted herewith be limited in breadth only by definition as defined in the specification and appended claims and any equivalents thereof.

1. A fluid removal device comprising:
an elongated body having a proximal end and a distal end;
a handle attached to the proximal end of the elongated body, the handle having one or more connections for coupling to one or more fluid or suction sources;
an absorbent material; and
a centrally located channel passing through at least a portion of the elongated body, wherein the absorbent material is positioned over a distal opening of the channel and extends proximally along a wall of the elongated body, and wherein the absorbent material and the channel are positionable in a first configuration to provide a first fluid flow rate through the absorbent material and the channel, and are movable with respect to one another to a second position to provide a second fluid flow rate through the channel.

2. The device of claim 1, wherein the absorbent covers an entire distal surface of the channel.

3. The device of either of claim 1 or 2, wherein the channel includes a distal opening, and wherein the absorbent material is positioned to completely cover a fluid flow path through the distal opening.

4. The device of claim 1, wherein the absorbent material extends at least half way along the distal end of the elongated body.

5. The device of claim 4, wherein the elongated body comprises one or more openings.

6. The device of claim 4, wherein the elongated body comprises a group of openings extending from a surface in fluid communication with the absorbent material into the channel.

7. The device of any of claim 5-6, wherein the absorbent material completely surrounds the outer surface of a distal length of the elongated body.

8. The device of claim 5-6, wherein the openings comprise holes or elongated slits.

9. The device of any of claims 1-8, wherein the elongate body comprises a first inner wall comprising one more openings extending through the inner wall and into the channel, and a second outer wall comprising one or more openings through the outer wall.

10. The device of claim 9, wherein the absorbent material is attached to a portion of an outer surface of the outer wall of the elongated body.

11. The device of claim 9, wherein the absorbent material is attached to a portion of an inner surface of the outer wall of the elongated body.

12. The device of any of claims 10, wherein the outer wall is longitudinally mobile with respect to the inner wall.

13. The device of claim 12, wherein the absorbent material is configured to be retracted to expose one or more openings in the inner wall.

14. The device of any of claims 9-10, wherein the inner wall is longitudinally translatable within the elongated body.

15. The device of any of claims 12-14, wherein the absorbent material comprises a distal region configured to allow passage of the inner wall therethrough.

16. The device of any one of claims 1-15, wherein the handle region further comprising an irrigation port.

17. The device of any one of claims 1-16, further comprising at least one additional channel passing through at least a portion of the elongated body.

18. The device of claim 17, wherein the first channel is in fluid communication with a negative pressure source and the at least one additional channel is in fluid communication with an irrigation source.

19. The device of any one of claims 1-18, wherein the elongated body has a cylindrical shape.

20. The device of any one of claims 1-19, wherein the channel has an asymmetric tubular shape.

21. The device of claim 20, wherein the asymmetric shape include a wave-like configuration and the channel wall is movable with respect to the absorbent body.

22. The device of claim 9, wherein the absorbent material body is movable along an outer surface of the inner wall.

23. The device of claim 22, wherein the absorbent material body forms a loop in a longitudinal direction along at least a portion of the inner wall.

24. The device of claim 22, wherein the absorbent material body is movable in a longitudinal direction with respect to the inner wall.

25. The device of any of claims 1-24, further comprising at least one treatment instrument positioned within the channel.

26. The device of claim 25, wherein the treatment instrument is longitudinally movable within the channel to allow the treatment instrument to be extended from the distal end of the channel.

27. The device of claim 26, wherein the treatment instrument is moveable to extend through the distal end of the channel.

28. The device of any one of claims 25-27, wherein the treatment instrument comprises an electrosurgical instrument.

29. The device of claim 28, wherein the treatment instrument is a monopolar instrument.

30. The device of any one of claims 25-27, wherein the instrument comprises a cutting instrument.

31. The device of any one of claims 25-27, wherein the instrument comprises a grasping instrument.

32. The device of any one of claims 25-27, wherein the instrument comprises a fastening instrument.

33. The device of any one of claims 1-32, wherein the handle comprises at least one actuator configured to control the negative pressure source.

34. The device of any one of claims 1-33, wherein the handle further comprises an actuator for switching the device between a configuration selected to provide the first flow rate and a configuration selected to provide the second flow rate.

35. The device of any one of claims 25-34, further comprising one or more controls for controlling operation of the treatment instrument.

36. The device of claim 35, wherein the one or more controls are positioned on the handle.

37. The device of claim 35, wherein the one or more controls comprise at least one control configured for operation by a foot pedal or button.

38. The device of any one of claims 1-37, wherein the absorbent material comprises a fabric.

39. The device of claim 38, wherein the fabric comprises surgical gauze.

40. The device of any one of claims 1-39, wherein the absorbent material comprises a sponge.

41. The device of any one of claims 1-40, wherein the absorbent material comprises a synthetic polymeric material.

42. A fluid removal device comprising:
a tubular body having a proximal end, a distal end, and a centrally located fluid passage extending from an opening at the distal end and at least partially through the tubular body towards the proximal end;
a handle attached to the proximal end of the tubular body, the handle having an actuator and a port for coupling to a negative pressure source; and
an absorbent material that is movable with respect to the tubular body between a first position and a second position, wherein the absorbent material is positioned over a distal opening of the fluid passage and extends proximally along a wall of the elongated body, wherein in the first position the absorbent material is positioned along a fluid path through the opening at the distal end of the tubular body to provide a first fluid flow rate through the absorbent material and fluid passage, and a second position to provide a second fluid flow rate through the fluid passage of the tubular body without passage of fluid through the absorbent material.

43. The device of claim 42, wherein the absorbent material covers an entire distal surface of the tubular body.

44. The device of either of claim 42 or 43, wherein the absorbent material is positioned to completely cover a fluid flow path through a distal opening of the tubular body.

45. The device of claim 44, wherein the absorbent material extends at least half way along the proximal end of the tubular body.

46. The device of claim 45, wherein the tubular body comprises one or more openings along its side surface.

47. The device of claim 46, wherein the tubular body comprises a group of openings.

48. The device of claim 47, wherein the openings comprise holes or elongated slits.

49. The device of any of claim 42-48, wherein the absorbent material completely surrounds an outer surface of a distal length of the elongated body.

50. The device of any of claims 42-49, wherein the fluid passage comprises an annular wall that is movable within the elongated body.

51. The device of any of claims 42-50, wherein the fluid passage is longitudinally translatable within the elongated body.

52. The device of claim 51, wherein the absorbent material comprises a distal region configured to allow passage of the fluid channel therethrough.

53. The device of claim 52, wherein the distal region comprises one or more flexible slits or flaps.

54. The device of any of claims 42-53, wherein the absorbent material is movable with respect to the tubular body.

55. The device of claim 54, wherein the absorbent material is configured to be retracted to allow fluid passage through a distal opening in the elongated body.

56. The device of claim 55, wherein the absorbent material is positioned on an outer surface of a movable tubular body.

57. The device of claim 55, wherein the absorbent material is positioned on an inner surface of a movable tubular body.

58. The device of any one of claims 42-57, wherein the handle region further comprises an irrigation port.

59. The device of any one of claims 42-58, further comprising at least one additional fluid channel passing through at least a portion of the elongated body.

60. The device of claim 59, wherein the first fluid channel is in fluid communication with a negative pressure source and the at least one additional fluid channel is in fluid communication with an irrigation source.

61. The device of any of claims 42-60, further comprising at least one treatment instrument positioned within the elongated body.

62. The device of claim 61, wherein the treatment instrument is longitudinally movable within the elongated body to allow the treatment instrument to be extended from the distal end of the elongated body.

63. The device of claim 61, wherein the elongated body is configured to be retracted to expose the treatment instrument through the distal end of the elongated body.

64. The device of any one of claims 61-63, wherein the treatment instrument comprises a cautery instrument.

65. The device of claim 64, wherein the treatment instrument is an electrocautery instrument.

66. The device of any one of claims 61-63, wherein the instrument comprises a cutting instrument.

67. The device of any one of claims 61-63, wherein the instrument comprises a grasping instrument.

68. The device of any one of claims 61-63, wherein the instrument comprises a fastening instrument.

69. The device of any one of claims 42-68, wherein the handle comprises at least one actuator configured to control the negative pressure source.

70. The device of any one of claims 43-68 wherein the handle further comprises an actuator for switching the device between a configuration selected to provide the first flow rate and a configuration selected to provide the second flow rate.

71. The device of any one of claims 61-68, further comprising one or more controls for controlling operation of the treatment instrument.

72. The device of claim 71, wherein the one or more controls are positioned on the handle.

73. The device of claim 71, wherein the one or more controls comprise at least one control configured for operation by a foot pedal or button.

74. The device of any one of claims 42-73, wherein the absorbent material comprises a fabric.

75. The device of claim 74, wherein the fabric comprises surgical gauze.

76. The device of any one of claims 42-73, wherein the absorbent material comprises a sponge.

77. The device of any one of claims 42-73, wherein the absorbent material comprises a synthetic polymeric material.

78. A method of removing fluid, the method comprising:
positioning a suction device within a surgical site, the device including a tubular body with a centrally located fluid channel and a movable absorbent material positioned over a distal opening in the fluid channel and extending proximally along a length of the tubular body;
applying negative pressure to the absorbent material to remove fluid from the surgical site through the absorbent material and the tubular body fluid channel at a first flow rate; and
adjusting the positions of the absorbent material and the tubular body with respect to one another to provide fluid communication between a distal opening in the tubular body and the fluid channel of the tubular body to remove fluid from the surgical site at a second flow rate through the fluid channel that is higher than the first flow rate.

79. The method of claim 78, wherein adjusting the configuration of the absorbent material relative to the tubular body comprises moving the absorbent material longitudinally within the tubular body.

80. The method of claim 78, wherein adjusting the configuration of the absorbent material relative to the tubular body comprises extending a fluid channel contained within the tubular body to expose an opening in the fluid channel for collection of fluids without passing the fluids through the absorbent material.

81. The method of any one of claims 78-80, further comprising inserting a port into a patient's abdomen and inserting the tubular body through the trocar such that a distal end portion of the tubular body extends into an abdominal cavity.

82. The method of any of claims 78-81, further comprising adjusting the flow rate between at least three different levels during a surgical procedure.

83. The method of any of claims 78-82, further comprising irrigating the surgical site with an irrigation fluid.

84. The method of any one of claims 78-83, further comprising contacting a tissue surface with the absorbent material.

85. The method of any one of claims 78-84, comprising suctioning fluid through a plurality of annular channels within the tubular body.

86. The method of any of claims 78-85, further comprising positioning a treatment instrument proximate the distal opening.

87. The method of claim 86, further comprising longitudinally moving the treatment instrument within the tubular body to allow the treatment instrument to be extended from the distal opening of the tubular body.

88. The method of claim 87, comprising retracting a portion of the tubular body to expose the treatment instrument through the distal opening of the elongated body.

89. The method of any one of claims 86-88, wherein the treatment instrument comprises an electrosurgical instrument.

90. The method of claim 89, wherein the treatment instrument is an electrocautery instrument.

91. The method of any one of claims 86-88, wherein the instrument comprises a cutting instrument.

92. The method of any one of claims 86-88, wherein the instrument comprises a grasping instrument.

93. The method of any one of claims 78-92, further comprising operating the handle comprises at least one actuator configured to control suction through the tubular body.

94. The method of any one of claims 78-92, further comprising operating one or more controls for controlling the treatment instrument.

95. The method of claim 94, wherein the one or more controls are positioned on the handle.

96. The method of claim 94, wherein the one or more controls comprise at least one control configured for operation by a foot pedal or button.

97. The method of any one of claims 78-96, wherein the absorbent material comprises a fabric.

98. The method of claim 97, wherein the fabric comprises surgical gauze.

99. The method of any one of claims 78-96, wherein the absorbent material comprises a sponge.

100. The method of any one of claims 78-96, wherein the absorbent material comprises a synthetic polymeric material.

101. A fluid removal device comprising:
an absorbent material; and
a flexible fluid conduit fluidly attached to the absorbent material, wherein a distal portion of the fluid conduit passes into an interior of the absorbent material, and wherein the distal portion comprises a group of openings in fluid communication with the absorbent material and a central lumen of the fluid conduit.

102. The device of claim 101, wherein the absorbent material comprises a cylindrical shape.

103. The device of either of claim 101 or 102, wherein the fluid conduit and absorbent material are configured to be securely coupled to one another.

104. The device of claim 103, wherein the fluid conduit and absorbent material include threaded connectors.

105. The device of claim 103, wherein the fluid conduit and absorbent material include a friction fit connection.

106. The device of claim 103, wherein one of the fluid conduit and the absorbent material comprises protruding tabs, and the other comprises locking slots for receiving the tabs.

107. The device of any one of claims 101-106, wherein the absorbent material comprises a fabric.

108. The device of claim 107, wherein the fabric comprises surgical gauze.

109. The device of any one of claims 101-106, wherein the absorbent material comprises a sponge.

110. The device of any one of claims 101-109, wherein the absorbent material comprises a synthetic polymeric material.

111. A laparoscopic surgical system, comprising:
a laparoscopic access port; and
a suction device, comprising:
an absorbent material; and
a flexible fluid conduit fluidly attached to the absorbent material, wherein a distal portion of the fluid conduit passes into an interior of the absorbent material, and wherein the distal portion comprises a group of openings in fluid communication with the absorbent material and a central lumen of the fluid conduit.

112. The system of claim 111, wherein the absorbent material comprises a cylindrical shape.

113. The system of either of claim 111 or 112, wherein the fluid conduit and absorbent material are configured to be securely coupled to one another.

114. The system of claim 113, wherein the fluid conduit and absorbent material include threaded connectors.

115. The system of claim 113, wherein the fluid conduit and absorbent material include a friction fit connection.

116. The system of claim 113, wherein one of the fluid conduit and the absorbent material comprises protruding tabs, and the other comprises locking slots for receiving the tabs.

117. The system of any one of claims 111-116, wherein the absorbent material comprises a fabric.

118. The system of claim 117, wherein the fabric comprises surgical gauze.

119. The system of any one of claims 111-116, wherein the absorbent material comprises a sponge.

120. The system of any one of claims 111-119, wherein the absorbent material comprises a synthetic polymeric material.

121. The system of any of claims 111-120, wherein the access port is a multi-lumen port.

122. The system of any of claims 111-120, wherein the access port comprises a single lumen configured to allow passage of multiple surgical instruments.

123. The system of any of claims 111-120, wherein the access port is a single-lumen port.

124. The system of any one of claims 111-123, wherein the access port further includes one or more attachment mechanisms for securing the fluid conduit.

125. A method of performing laparoscopic surgery, comprising:
forming an opening passing through an abdominal wall;
passing an absorbent material into an abdominal cavity, wherein the absorbent material is attached to a fluid conduit;
attaching an access port to the abdominal cavity; passing a surgical instrument through the access port; and
applying negative pressure to the fluid conduit to provide indirect suction.

126. The method of claim 125, further comprising passing the fluid conduit through a lumen of the access port.

127. The method of claim 126, wherein the fluid conduit is passed through the lumen before attaching the access port to the abdominal cavity.

128. The method of claim 126, wherein the access port has multiple lumens, and the fluid conduit and surgical instrument are passed through separate lumens.

129. The method of claim 125, wherein the fluid conduit is positioned adjacent the access port and passes through the opening in the abdominal wall.

130. The method of any of claims 125-129, wherein a distal portion of the fluid conduit passes into an interior of the absorbent material, and wherein the distal portion comprises a group of openings in fluid communication with the absorbent material and a central lumen of the fluid conduit.

131. The method of any of claims 125-129, wherein the absorbent material and fluid conduit are securely attached to one another, and the method further comprises removing the absorbent material and fluid conduit from the abdominal cavity at the same time.

132. A fluid removal device, comprising:
an absorbent material; and
a flexible fluid conduit fluidly attached to the absorbent material, wherein a distal portion of the fluid conduit passes into an interior of the absorbent material, the conduct comprising at least one opening in fluid communication with the absorbent material and a central lumen of the fluid conduit.

133. The device of claim 132, wherein the absorbent material comprises a cylindrical shape.

134. The device of any of claims 1-133, wherein the fluid conduit and absorbent material are configured to be securely coupled to one another.

135. The device of claim 134, wherein the fluid conduit and absorbent material include threaded connectors.

136. The device of claim 134, wherein the fluid conduit and absorbent material include a friction fit connection.

137. The device of claim 134, wherein one of the fluid conduit and the absorbent material comprises protruding tabs, and the other comprises locking slots for receiving the tabs.

138. The device of any one of claims 132-134, wherein the absorbent material comprises a fabric.

139. The device of claim 138, wherein the fabric comprises surgical gauze.

140. The device of any one of claims 132-134, wherein the absorbent material comprises a sponge such as a synthetic polymeric material.
